(19) United States
(12) Patent Application Publication
(10) Pub. No.: US 2011/0165144 A1
(43) Pub. Date: Jul. 7, 2011

(54) METHODS FOR TREATING RHEUMATOID ARTHRITIS

(75) Inventors: Bryce A. Binstadt, Stillwater, MN (US); Diane J. Mathis, Brookline, MA (US); Christophe O. Benoist, Brookline, MA (US)

(73) Assignee: JOSLIN DIABETES CENTER, INC., Boston, MA (US)

(21) Appl. No.: 12/377,538

(22) PCT Filed: Aug. 14, 2007
(86) PCT No.: PCT/US07/75876

§ 371 Date: Dec. 9, 2010

Related U.S. Application Data
(60) Provisional application No. 60/838,432, filed on Aug. 16, 2006.

(51) Int. Cl. A61K 39/395; A61K 31/7088; A61K 38/08; A61P 19/02; A61P 29/00; G01N 33/53; A61K 49/00

(52) U.S. Cl. 424/130.1; 514/44 R; 514/44 A; 514/16.6; 435/7.2; 800/3

(57) ABSTRACT

Provided are methods of treating rheumatoid arthritis by inhibiting Neuromedin U signalling.

### TECHNICAL FIELD

This invention relates to methods targeting neuromedin U (NMU) for the treatment of rheumatoid arthritis.

### BACKGROUND

Rheumatoid arthritis (RA) affects approximately 1% of the world's population, and is a significant cause of morbidity. The current therapy of RA is directed at suppressing the immune system, either by interference with the function of lymphocytes or cytokines. However, until a cure for RA is found, other molecules and pathways need to be considered as potential therapeutic targets.

### SUMMARY

The present invention is based, at least in part, on the discovery that the neuropeptide neuromedin U (NMU) plays a pivotal role in the pathogenesis of inflammatory arthritis in a mouse model. Therefore, pharmacologic blockade of this newly-recognized inflammatory pathway may be beneficial for patients with RA.

In one aspect, the invention provides methods for treating rheumatoid arthritis (RA) in an animal. The methods include administering a therapeutically effective amount of one or more of the following:

(i) a neuromedin U (NMU)-specific inhibitory nucleic acid, e.g., an siRNA, antisense, aptamer, or ribozyme targeted specifically to NMU;

(ii) a neuromedin U (NMU) inhibitory peptide, e.g., a peptide comprising the sequence Phe-Arg-Pro-Arg-Asn; or

(iii) an antibody or antigen binding fragment thereof that binds to an NMU-R, e.g., NMU-R1, and inhibits NMU signalling, e.g., inhibits binding of NMU to the NMU-R1.

In another aspect, the invention provides methods for identifying a candidate compound for the treatment of rheumatoid arthritis (RA). The methods include:
providing a sample comprising neuromedin U (NMU) and a NMU Receptor (NMU-R), e.g., NMU-R1 or NMU-R2;
contacting the sample with a test compound; and
evaluating binding of the NMU to the NMU-R in the sample in the presence and absence of the test compound.

A test compound that decreases binding of NMU to an NMU-R is a candidate compound for the treatment of RA.

In some embodiments, the sample includes a cell expressing the NMU-R, or membranes isolated from cells expressing the NMU-R.

In a further aspect, the invention provides methods for identifying a candidate agent for the treatment of RA. The methods include providing an experimental animal model of RA; inducing RA in the model; administering a candidate compound identified by the method of claim 2 to the animal before, during, and/or after inducing RA; and evaluating one or more clinical parameters of RA in the animal model. A candidate compound that is associated with an improvement, e.g., a statistically significant improvement, in a clinical parameter of RA is a candidate agent for the treatment of RA.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety, specifically including the priority provisional application, U.S. Ser. No. 60/838,432, filed on Aug. 16, 2006. In case of conflict, the present specification, including definitions, will control.

Other features and advantages of the invention will be apparent from the following detailed description and figures, and from the claims.

### DESCRIPTION OF DRAWINGS

FIG. 1 is a pair of line graphs showing that Neuromedin U gene knockout mice (NMU −/−) are resistant to serum transfer arthritis. Arthritis scores (maximum=12) and ankle measurements were obtained for two weeks. Values indicate mean±standard deviation; n=3 mice/group. Data are representative of two experiments with a total of 7 mice/group.

FIG. 2 is a set of four photomicrographs showing that NMU −/− mice show minimal joint inflammation but have normal mast cells. Toluidine blue staining demonstrates the presence of mast cells in NMU −/− mice (arrows in lower panels).

FIG. 3 is a line graph showing that the early vascular leak response was normal, suggesting that early activation of mast cells is intact. MFI=mean fluorescence intensity.

FIG. 4 is a line graph showing that NMU −/− mice maintain normal titers of arthritogenic antibodies.

### DETAILED DESCRIPTION

The neuropeptide neuromedin U (NMU) plays a pivotal role in the pathogenesis of inflammatory arthritis in a mouse model. Therefore, pharmacologic blockade of this newly-recognized inflammatory pathway may be beneficial for patients with RA.

Neuromedin U (NMU) and NMU Receptors (NMU-Rs)

NMU is a neuropeptide expressed in several tissues, including the gastrointestinal tract, central nervous system, bone marrow, skin, and other tissues. NMU mediates a variety of physiological functions, the best-characterized of which include the control of feeding behavior and regulation of muscle contraction. See, e.g., Brighton et al., Pharmacol. Rev. 56(2):231-48 (2004). Austin et al., J. Molec. Endocr. 14:157-169 (1995) isolated a human pituitary cDNA (GenBank Acc No. NM_{—}006681.1) encoding a deduced 174-amino acid NMU precursor that shares 70% amino acid sequence similarity with the rat NMU precursor (NP_{—}006672.1):

| (SEQ ID NO: 1) | |
|---|---|
| 1 | MLRTESCRPR SPAGQVAAAS PLLLLLLLLA WCAGACRGAP ILPQGLQPEQ QLQLWNEIDD |
| 61 | TCSSFLSIDS QPQASNALEE LCFMIMGMLP KPQEQDEKDN TKRFLFHYSK TQKLGKSNVV |
| 121 | SSVVHPLLQL VPHLHERRMK RFRVDEEFQS PFASQSRGYF LFRPRNGRRS AGFI |

The human NMU precursor contains a signal peptide and 4 paired basic residues, which are putative proteolytic processing sites, indicating that it may generate 3 peptides, including NMU. The 25-residue human NMU peptide is located near the C terminus of its precursor. The sequence is as follows:

| | |
|---|---|
| 1 FRVDEEFQSP FASQSRGYFL FRPRN 25 | (SEQ ID NO:2) |

The last five amino acids are absolutely conserved.

Two receptors for neuromedin U have been identified, termed NMU-R1 and NMU-R2, both of which are G protein coupled receptors. NMU-R1 is widely expressed, whereas the expression of NMU-R2 is localized predominantly to neural tissue. See, e.g., Howard et al., Nature 406:70-74 (2000); Brighton et al., 2004, supra. The sequences of human NMU-R1 and -R2 are publicly available in the Genbank database, see, e.g., NM_{—}006056.3 (NMU-R1 nucleic acid); NP_{—}006047.2 (NMU-R1 amino acid); NM_{—}020167.3 (NMU-R2 nucleic acid); and NP_{—}064552.2 (NMU-R2 amino acid).

The amino acid sequence of human NMU-R1 is as follows:

| (SEQ ID NO: 3) | |
|---|---|
| 1 | MTPLCLNCSV LPGDLYPGGA RNPMACNGSA ARGHFDPEDL NLTDEALRLK YLGPQQTELF |
| 61 | MPICATYLLI FVVGAVGNGL TCLVILRHKA MRTPTNYYLF SLAVSDLLVL LVGLPLELYE |
| 121 | MWHNYPFLLG VGGCYFRTLL FEMVCLASVL NVTALSVERY VAVVHPLQAR SMVTRAHVRR |
| 181 | VLGAVWGLAM LCSLPNTSLH GIQQLHVPCR GPVPDSAVCM LVRPRALYNM VVQTTALLFF |
| 241 | CLPMAIMSVL YLLIGLRLRR ERLLLMQEAK GRGSAAARSR YTCRLQQHDR GRRQVTKMLF |
| 301 | VLVVVFGICW APFHADRVMW SVVSQWTDGL HLAFQHVHVI SGIFFYLGSA ANPVLYSLMS |
| 361 | SRFRETFQEA LCLGACCHRL RPRHSSHSLS RMTTGSTLCD VGSLGSWVHP LAGNDGPEAQ |
| 421 | QETDPS |

The amino acid sequence of human NMU-R2 is as follows:

| (SEQ ID NO: 4) | |
|---|---|
| 1 | MSGMEKLQNA SWIYQQKLED PFQKHLNSTE EYLAFLCGPR RSHFFLPVSV VYVPIFVVGV |
| 61 | IGNVLVCLVI LQHQAMKTPT NYYLFSLAVS DLLVLLLGMP LEVYEMWRNY PFLFGPVGCY |
| 121 | FKTALFETVC FASILSITTV SVERYVAILH PFRAKLQSTR RRALRILGIV WGFSVLFSLP |
| 181 | NTSIHGIKFH YFPNGSLVPG SATFTVIKPM WIYNFIIQVT SFLFYLLPMT VISVLYYLMA |
| 241 | LRLKKDKSLE ADEGNANIQR PCRKSVNKML FVLVLVFAIC WAPFHIDRLF FSFVEEWSES |
| 301 | LAAVFNLVHV VSGVFFYLSS AVNPIIYNLL SRRFQAAFQN VISSFHKQWH SQHDPQLPPA |
| 361 | QRNIFLTECH FVELTEDIGP QFPCQSSMHN SHLPTALSSE QMSRTNYQSF HFNKT |

A role for NMU in control of feeding behavior was suggested by the finding that mice lacking the gene encoding NMU develop obesity, increased adiposity, and decreased activity. These NMU-deficient mice have subsequently been used to demonstrate a potential role for NMU in the regulation of inflammation. In particular, NMU directly activates mast cells, a cell type critical for the development of arthritis in the K/BxN serum transfer system. This activation likely occurs via NMU-R1 expressed on mast cells. Further studies have demonstrated that NMU-deficient mice are resistant to cecal-ligation puncture and LPS-induced septic shock, possibly due to decreased production of the cytokine interleukin-6. In a model of allergen-induced asthma, primary immune responses to the allergen were intact in NMU-deficient mice, whereas the influx of eosinophils into the lung was dramatically decreased. Thus, although NMU was originally identified as a peptide controlling feeding and muscle contraction, recent evidence suggests that NMU also plays a pivotal role in the development of inflammatory processes.

In the methods described herein, the sequences of NMU or an NMU-R set forth herein can be used. Alternatively, in some embodiments, sequences that are at least about 80%, 85%, 90%, 91%, 92%, 93%, 94%, 95%, 96%, 97%, 98% or 99% or more identical to a sequence described herein can be used. The comparison of sequences and determination of percent identity between two sequences can be accomplished using a mathematical algorithm. For the methods described herein, the percent identity between two amino acid sequences is determined using the Needleman and Wunsch ((1970) J. Mol. Biol. 48:444-453) algorithm which has been incorporated into the GAP program in the GCG software package (available on the world wide web at gcg.com), using the default parameters, i.e., a Blossum 62 scoring matrix with a gap penalty of 12, a gap extend penalty of 4, and a frameshift gap penalty of 5.

Sequences from other species can also be used, e.g., Pan troglodytes (XP_{—}001142975.1, NMU; XP_{—} 001140471.1, NMUR1; XP_{—}527091.1, NMU-R2); Mus musculus (NP_{—}062388.1, NMU; NP_{—}034471.1, NMU-R1; NP_{—}694719.2, NMU-R2); or Rattus norvegicus (NP_{—}071575.1, NMU; NP_{—}075588.1, NMU-R1; NP 071611.2, NMU-R2).

Inhibitory Nucleic Acid Molecules

The present invention also includes methods for treating, preventing, or delaying the onset of RA by administering inhibitory nucleic acid molecules that are targeted to a NMU or an NMU-R, e.g., NMU-R1RNA, e.g., antisense, siRNA, ribozymes, and aptamers.

siRNA Molecules

RNAi is a process whereby double-stranded RNA (dsRNA, also referred to herein as si RNAs or ds siRNAs, for double-stranded small interfering RNAs,) induces the sequence-specific degradation of homologous mRNA in animals and plant cells (Hutvagner and Zamore, Curr. Opin. Genet. Dev.:12, 225-232 (2002); Sharp, Genes Dev., 15:485-490 (2001)). In mammalian cells, RNAi can be triggered by 21-nucleotide (nt) duplexes of small interfering RNA (siRNA) (Chiu et al., Mol. Cell. 10:549-561 (2002); Elbashir et al., Nature 411:494-498 (2001)), or by micro-RNAs (miRNA), functional small-hairpin RNA (shRNA), or other dsRNAs which are expressed in vivo using DNA templates with RNA polymerase III promoters (Zeng et al., Mol. Cell. 9:1327-1333 (2002); Paddison et al., Genes Dev. 16:948-958 (2002); Lee et al., Nature Biotechnol. 20:500-505 (2002); Paul et al., Nature Biotechnol. 20:505-508 (2002); Tuschl, Nature Biotechnol. 20:440-448 (2002); Yu et al., Proc. Natl. Acad. Sci. USA 99(9):6047-6052 (2002); McManus et al., RNA 8:842-850 (2002); Sui et al., Proc. Natl. Acad. Sci. USA 99(6):5515-5520 (2002)).

The nucleic acid molecules or constructs can include dsRNA molecules comprising 16-30, e.g., 16, 17, 18, 19, 20, 21, 22, 23, 24, 25, 26, 27, 28, 29, or 30 nucleotides in each strand, wherein one of the strands is substantially identical, e.g., at least 80% (or more, e.g., 85%, 90%, 95%, or 100%) identical, e.g., having 3, 2, 1, or 0 mismatched nucleotide(s), to a target region in the mRNA, and the other strand is complementary to the first strand. The dsRNA molecules can be chemically synthesized, or can transcribed be in vitro from a DNA template, or in vivo from, e.g., shRNA. The dsRNA molecules can be designed using any method known in the art; a number of algorithms are known, and are commercially available. Gene walk methods can be used to optimize the inhibitory activity of the siRNA.

A number of effective NMU siRNA molecules are known in the art, and several are commercially available. See, e.g., Santa Cruz Biotechnology, Inc. (NMU-23 siRNA); Origene (HuSH 29mer shRNA Constructs against NMU); and Ambion/Applied Biosystems (NMU siRNA targeting exon 4). One method for preparing NMU siRNA is described in Shetzline et al., Blood, 104(6):1833-1840 (2004).

The nucleic acid compositions can include both siRNA and modified siRNA derivatives, e.g., siRNAs modified to alter a property such as the pharmacokinetics of the composition, for example, to increase half-life in the body, as well as engineered RNAi precursors.

siRNAs can be delivered into cells by methods known in the art, e.g., cationic liposome transfection and electroporation. siRNA duplexes can be expressed within cells from engineered RNAi precursors, e.g., recombinant DNA constructs using mammalian Pol III promoter systems (e.g., H1 or U6/snRNA promoter systems (Tuschl (2002), supra) capable of expressing functional double-stranded siRNAs; (Bagella et al., J. Cell. Physiol. 177:206-213 (1998); Lee et al. (2002), supra; Miyagishi et al. (2002), supra; Paul et al. (2002), supra; Yu et al. (2002), supra; Sui et al. (2002), supra). Transcriptional termination by RNA Pol III occurs at runs of four consecutive T residues in the DNA template, providing a mechanism to end the siRNA transcript at a specific sequence. The siRNA is complementary to the sequence of the target gene in 5′-3′ and 3′-5′ orientations, and the two strands of the siRNA can be expressed in the same construct or in separate constructs. Hairpin siRNAs, driven by H1 or U6 snRNA promoter and expressed in cells, can inhibit target gene expression (Bagella et al. (1998), supra; Lee et al. (2002), supra; Miyagishi et al. (2002), supra; Paul et al. (2002), supra; Yu et al. (2002), supra; Sui et al. (2002) supra). Constructs containing siRNA sequence under the control of T7 promoter also make functional siRNAs when cotransfected into the cells with a vector expression T7 RNA polymerase (Jacque (2002), supra).

Antisense

An “antisense” nucleic acid can include a nucleotide sequence that is complementary to a “sense” nucleic acid encoding a protein, e.g., complementary to the coding strand of a double-stranded cDNA molecule or complementary to an NMU or NMU-R mRNA sequence. The antisense nucleic acid can be complementary to an entire coding strand of a target sequence, or to only a portion thereof. In another embodiment, the antisense nucleic acid molecule is antisense to a “noncoding region” of the coding strand of a nucleotide sequence (e.g., the 5′ and 3′ untranslated regions).

An antisense nucleic acid can be designed such that it is complementary to the entire coding region of a target mRNA, but can also be an oligonucleotide that is antisense to only a portion of the coding or noncoding region of the target mRNA. For example, the antisense oligonucleotide can be complementary to the region surrounding the translation start site of the target mRNA, e.g., between the −10 and +10 regions of the target gene nucleotide sequence of interest. An antisense oligonucleotide can be, for example, about 7, 10, 15, 20, 25, 30, 35, 40, 45, 50, 55, 60, 65, 70, 75, 80, or more nucleotides in length.

An antisense nucleic acid can be constructed using chemical synthesis and enzymatic ligation reactions using procedures known in the art. For example, an antisense nucleic acid (e.g., an antisense oligonucleotide) can be chemically synthesized using naturally occurring nucleotides or variously modified nucleotides designed to increase the biological stability of the molecules or to increase the physical stability of the duplex formed between the antisense and sense nucleic acids, e.g., phosphorothioate derivatives and acridine substituted nucleotides can be used. The antisense nucleic acid also can be produced biologically using an expression vector into which a nucleic acid has been subcloned in an antisense orientation (i.e., RNA transcribed from the inserted nucleic acid will be of an antisense orientation to a target nucleic acid of interest, described further in the following subsection).

Based upon the sequences disclosed herein, one of skill in the art can easily choose and synthesize any of a number of appropriate antisense molecules for use in accordance with the present invention. For example, a “gene walk” comprising a series of oligonucleotides of 15-30 nucleotides spanning the length of a target nucleic acid can be prepared, followed by testing for inhibition of target gene expression. Optionally, gaps of 5-10 nucleotides can be left between the oligonucleotides to reduce the number of oligonucleotides synthesized and tested.

In some embodiments, the antisense nucleic acid molecule is an α-anomeric nucleic acid molecule. An α-anomeric nucleic acid molecule forms specific double-stranded hybrids with complementary RNA in which, contrary to the usual β-units, the strands run parallel to each other (Gaultier et al., Nucleic Acids. Res. 15:6625-6641 (1987)). The antisense nucleic acid molecule can also comprise a 2′-o-methylribonucleotide (Inoue et al. Nucleic Acids Res. 15:6131-6148 (1987)) or a chimeric RNA-DNA analogue (Inoue et al. FEBS Lett., 215:327-330 (1987)).

In some embodiments, the antisense nucleic acid is a morpholino oligonucleotide (see, e.g., Heasman, Dev. Biol. 243:209-14 (2002); Iversen, Curr. Opin. Mol. Ther. 3:235-8 (2001); Summerton, Biochim. Biophys. Acta. 1489:141-58 (1999).

Target gene expression can be inhibited by targeting nucleotide sequences complementary to a regulatory region (e.g., promoters and/or enhancers) to form triple helical structures that prevent transcription of the SptS gene in target cells. See generally, Helene, Anticancer Drug Des. 6:569-84 (1991); Helene, Ann. N.Y. Acad. Sci. 660:27-36 (1992); and Maher, Bioassays 14:807-15 (1992). The potential sequences that can be targeted for triple helix formation can be increased by creating a so called “switchback” nucleic acid molecule. Switchback molecules are synthesized in an alternating 5′-3′,3′-5′ manner, such that they base pair with first one strand of a duplex and then the other, eliminating the necessity for a sizeable stretch of either purines or pyrimidines to be present on one strand of a duplex.

Ribozymes

Ribozymes are a type of RNA that can be engineered to enzymatically cleave and inactivate other RNA targets in a specific, sequence-dependent fashion. By cleaving the target RNA, ribozymes inhibit translation, thus preventing the expression of the target gene. Ribozymes can be chemically synthesized in the laboratory and structurally modified to increase their stability and catalytic activity using methods known in the art. Alternatively, ribozyme genes can be introduced into cells through gene-delivery mechanisms known in the art. A ribozyme having specificity for a target nucleic acid can include one or more sequences complementary to the nucleotide sequence of a cDNA described herein, and a sequence having known catalytic sequence responsible for mRNA cleavage (see U.S. Pat. No. 5,093,246 or Haselhoff and Gerlach Nature 334:585-591 (1988)). For example, a derivative of a Tetrahymena L-19 IVS RNA can be constructed in which the nucleotide sequence of the active site is complementary to the nucleotide sequence to be cleaved in a target mRNA. See, e.g., Cech et al. U.S. Pat. No. 4,987,071; and Cech et al. U.S. Pat. No. 5,116,742. Alternatively, a target mRNA can be used to select a catalytic RNA having a specific ribonuclease activity from a pool of RNA molecules. See, e.g., Bartel and Szostak, Science 261:1411-1418 (1993).

Aptamers

Aptamers are short oligonucleotide sequences which can specifically bind specific proteins. It has been demonstrated that different aptameric sequences can bind specifically to different proteins, for example, the sequence GGNNGG where N=guanosine (G), cytosine (C), adenosine (A) or thymidine (T) binds specifically to thrombin (Bock et al (1992) Nature 355: 564 566 and U.S. Pat. No. 5,582,981 (1996) Toole et al). Methods for selection and preparation of such RNA aptamers are knotn in the art (see, e.g., Famulok, Curr. Opin. Struct. Biol. 9:324 (1999); Herman and Patel, J. Science 287:820-825 (2000)); Kelly et al., J. Mol. Biol. 256:417 (1996); and Feigon et al., Chem. Biol. 3: 611 (1996)).

Administration of Inhibitory Nucleic Acid Molecules

The inhibitory nucleic acid molecules described herein can be administered to a subject (e.g., by direct injection at a tissue site), or generated in situ such that they hybridize with or bind to cellular mRNA and/or genomic DNA encoding a target protein to thereby inhibit expression of the protein, e.g., by inhibiting transcription and/or translation. Alternatively, inhibitory nucleic acid molecules can be modified to target selected cells and then administered systemically. For systemic administration, inhibitory nucleic acid molecules can be modified such that they specifically bind to receptors or antigens expressed on a selected cell surface, e.g., by linking the inhibitory nucleic acid nucleic acid molecules to peptides or antibodies that bind to cell surface receptors or antigens. The inhibitory nucleic acid nucleic acid molecules can also be delivered to cells using the vectors described herein. To achieve sufficient intracellular concentrations of the inhibitory nucleic acid molecules, vector constructs in which the inhibitory nucleic acid nucleic acid molecule is placed under the control of a strong promoter can be used.

NMU Inhibitory Peptides

The present invention also includes methods for treating, preventing, or delaying the onset of RA by administering inhibitory NMU peptides. In some embodiments, the NMU inhibitory peptides include the sequence FRPRN (SEQ ID NO:5). As this sequence is conserved across species, it is expected that such a peptide will have inhibitory properties. In some embodiments, the methods include administering an inhibitory peptide analog of NMU that includes the sequence FRPRN (SEQ ID NO:5) and one or more, e.g., 2, 3, 4, 5, 10 or more, non-conservative substitutions or deletions outside the sequence FRPRN (SEQ ID NO:5). In other embodiments, the methods include administering an inhibitory peptide analog that includes one or more non-conservative substitutions in the sequence FRPRN (SEQ ID NO:5). Such inhibitory peptides and analogs can be modified as described herein to alter one or more pharmacokinetic properties and/or therapeutic activity.

Anti-NMU Receptor Inhibitory Antibodies

In some embodiments, the methods described herein include administering an anti-NMU-R antibody, e.g., an anti-NMU-R1 antibody, that inhibits NMU signalling through the NMU-R, e.g., prevents binding of NMU to the NMU-R, i.e., acts as a competitive inhibitor. The term “antibody” as used herein refers to an immunoglobulin molecule or antigen-binding portion thereof. Examples of antigen-binding portions of immunoglobulin molecules include F(ab) and F(ab′)₂ fragments which can be generated by treating the antibody with an enzyme such as pepsin.

The antibody can be a polyclonal, monoclonal, recombinant, e.g., a chimeric or humanized, fully human, non-human, e.g., murine, or single chain antibody. In some embodiments it has no effector function and cannot fix complement.

A full-length NMU-R protein or antigenic peptide fragment of NMU-R, e.g., the extracellular can be used as an immunogen or can be used to identify anti-NMU-R antibodies made with other immunogens, e.g., cells, membrane preparations, and the like. The antigenic peptide of NMU-R should include at least 8 amino acid residues of an extracellular sequence. Preferably, the antigenic peptide includes at least 10 amino acid residues, more preferably at least 15 amino acid residues, even more preferably at least 20 amino acid residues, and most preferably at least 30 amino acid residues.

Fragments of human NMU-R1 that include residues about 1-65, 119-138, 203-235, or 316-338 of GenBank Accession Sequence NP_{—}006047.2 (encoded by nucleic acid sequence NM_{—}006056.2) can be used as immunogens or used to characterize the specificity of an antibody.

In a preferred embodiment the antibody can bind to an extracellular portion of the human NMU-R1 protein, and prevents binding of NMU to the receptor.

In some embodiments, the antibody binds to the NMU-R2 receptor, e.g., an extracellular portion of the human NMU-R2 protein.

Chimeric, humanized, and completely human antibodies are desirable for applications which include repeated administration, e.g., therapeutic treatment (and some diagnostic applications) of human patients.

The anti-NMU-R antibody can be a single chain antibody. A single-chain antibody (scFV) may be engineered (see, for example, Colcher, D. et al. (1999) Ann N Y Acad Sci 880:263-80; and Reiter, Y. (1996) Clin Cancer Res 2:245-52). The single chain antibody can be dimerized or multimerized to generate multivalent antibodies having specificities for different epitopes of the same target NMU-R protein.

In a preferred embodiment, the antibody has reduced or no ability to bind an Fc receptor. For example., it is a isotype or subtype, fragment or other mutant, which does not support binding to an Fc receptor, e.g., it has a mutagenized or deleted Fc receptor binding region.

Pharmacokinetic Properties and Therapeutic Activity

In some embodiments, the therapeutic agent is a protein, e.g., a peptide or polypeptide. Modifications can be made to a protein to alter the pharmacokinetic properties of the protein to make it more suitable for use in protein therapy. For example, such modifications can result in longer circulatory half-life, an increase in cellular uptake, improved distribution to targeted tissues, a decrease in clearance and/or a decrease of immunogenicity. A number of approaches useful to optimize the therapeutic activity of a protein, e.g., a therapeutic protein described herein, e.g., a NMU peptide or analog thereof, or a protein that inhibits NMU signalling activity, are known in the art, including chemical modification.

Expression System

For recombinant proteins, the choice of expression system can influence pharmacokinetic characteristics. Differences in post-translational processing between expression systems can lead to recombinant proteins of varying molecular size and charge, which can affect circulatory half-life, rate of clearance and immunogenicity, for example. The pharmacokinetic properties of the protein may be optimized by the appropriate selection of an expression system, such as selection of a bacterial, viral, or mammalian expression system. Exemplary mammalian cell lines useful in expression systems for therapeutic proteins are Chinese hamster ovary, (CHO) cells, the monkey COS-1 cell line and the CV-1 cell line.

Chemical Modification

A protein can be chemically altered to enhance the pharmacokinetic properties, while maintaining activity. The protein can be covalently linked to a variety of moieties, altering the molecular size and charge of the protein and consequently its pharmacokinetic characteristics. The moieties are preferably non-toxic and biocompatible. In one embodiment, poly-ethylene glycol (PEG) can be covalently attached to the protein (PEGylation). A variety of PEG molecules are known and/or commercially available (See, e.g., Sigma-Aldrich catalog). PEGylation can increase the stability of the protein, decrease immunogenicity by steric masking of epitopes, and improve half-life by decreasing glomerular filtration. (See, e.g., Harris and Zalipsky, Poly(ethylene glycol): Chemistry and Biological Applications, ACS Symposium Series, No. 680, American Chemical Society (1997); Harris et al., Clinical Pharmacokinetics, 40:7, 485-563 (2001)). Examples of therapeutic proteins administered as PEG constructs include Adagen™ (PEG-ADA) and Oncospar™ (Pegylated asparaginase). In another embodiment, the protein can be similarly linked to oxidized dextrans via an amino group. (See Sheffield, Curr. Drug Targets Cardiovas. Haemat. Dis., 1:1, 1-22 (2001)). In yet another embodiment, conjugation of arginine oligomers to cyclosporin A can facilitates topical delivery (Rothbard et al., Nat. Med., 6(11):1253-1257 (2000)).

Furthermore, the therapeutic protein can be chemically linked to another protein, e.g., cross-linked (via a bifunctional cross-linking reagent, for example) to a carrier protein to form a larger molecular weight complex with longer circulatory half-life and improved cellular uptake. In some embodiments, the carrier protein can be a serum protein, such as albumin. In another embodiment, the therapeutic protein can cross-link with itself to form a homodimer, a trimer, or a higher analog, e.g., via heterobifunctional or homobifunctional cross-linking reagents (see Stykowski et al., Proc. Natl. Acad. Sci. USA, 95:1184-1188 (1998)). Increasing the molecular weight and size of the therapeutic protein through dimerization or trimerization can decrease clearance.

Modification of Protein Formulation

The formulation of the protein may also be changed. For example, the therapeutic protein can be formulated in a carrier system. The carrier can be a colloidal system. The colloidal system can be a liposome, a phospholipid bilayer vehicle. In one embodiment, the therapeutic protein is encapsulated in a liposome while maintaining protein integrity. As one skilled in the art would appreciate, there are a variety of methods to prepare liposomes. (See Lichtenberg et al., Methods Biochem. Anal., 33:337-462 (1988); Anselem et al., Liposome Technology, CRC Press (1993)). Liposomal formulations can delay clearance and increase cellular uptake (See Reddy, Ann. Pharmacother., 34 (7-8):915-923 (2000)).

The carrier can also be a polymer, e.g., a biodegradable, biocompatible polymer matrix. In one embodiment, the therapeutic protein can be embedded in the polymer matrix, while maintaining protein integrity. The polymer may be natural, such as polypeptides, proteins or polysaccharides, or synthetic, such as poly(α-hydroxy) acids. Examples include carriers made of, e.g., collagen, fibronectin, elastin, cellulose acetate, cellulose nitrate, polysaccharide, fibrin, gelatin, and combinations thereof. In one embodiment, the polymer is poly-lactic acid (PLA) or copoly lactic/glycolic acid (PGLA). The polymeric matrices can be prepared and isolated in a variety of forms and sizes, including microspheres and nanospheres. Polymer formulations can lead to prolonged duration of therapeutic effect. (See Reddy, Ann. Pharmacother., 34 (7-8):915-923 (2000)). A polymer formulation for human growth hormone (hGH) has been used in clinical trials. (See Kozarich and Rich, Chemical Biology, 2:548-552 (1998)).

Examples of polymer microsphere sustained release formulations are described in PCT publication WO 99/15154 (Tracy et al.), U.S. Pat. Nos. 5,674,534 and 5,716,644 (both to Zale et al.), PCT publication WO 96/40073 (Zale et al.), and PCT publication WO 00/38651 (Shah et al.). U.S. Pat. Nos. 5,674,534 and 5,716,644 and PCT publication WO 96/40073 describe a polymeric matrix containing particles of erythropoietin that are stabilized against aggregation with a salt.

Pharmaceutical Compositions

The methods described herein include the manufacture and use of pharmaceutical compositions, which include NMU inhibitory nucleic acids and peptides, as well as compounds identified by a method described herein, as active ingredients. Also included are the pharmaceutical compositions themselves.

Pharmaceutical compositions typically include a pharmaceutically acceptable carrier. As used herein the language “pharmaceutically acceptable carrier” includes saline, solvents, dispersion media, coatings, antibacterial and antifungal agents, isotonic and absorption delaying agents, and the like, compatible with pharmaceutical administration. Supplementary active compounds can also be incorporated into the compositions.

Pharmaceutical compositions are typically formulated to be compatible with its intended route of administration. Examples of routes of administration include parenteral, e.g., intravenous, intradermal, subcutaneous, oral (e.g., inhalation), transdermal (topical), transmucosal, and rectal administration.

Methods of formulating suitable pharmaceutical compositions are known in the art, see, e.g., the books in the series Drugs and the Pharmaceutical Sciences: a Series of Textbooks and Monographs (Dekker, N.Y.). For example, solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use can include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringability exists. It should be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, sodium chloride in the composition. Prolonged absorption of the injectable compositions can be brought about by including in the composition an agent that delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle, which contains a basic dispersion medium and the required other ingredients from those enumerated above. In the case of sterile powders for the preparation of sterile injectable solutions, the preferred methods of preparation are vacuum drying and freeze-drying, which yield a powder of the active ingredient plus any additional desired ingredient from a previously sterile-filtered solution thereof.

Oral compositions generally include an inert diluent or an edible carrier. For the purpose of oral therapeutic administration, the active compound can be incorporated with excipients and used in the form of tablets, troches, or capsules, e.g., gelatin capsules. Oral compositions can also be prepared using a fluid carrier for use as a mouthwash. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds can be delivered in the form of an aerosol spray from a pressured container or dispenser that contains a suitable propellant, e.g., a gas such as carbon dioxide, or a nebulizer. Such methods include those described in U.S. Pat. No. 6,468,798.

Systemic administration of a therapeutic compound as described herein can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The pharmaceutical compositions can also be prepared in the form of suppositories (e.g., with conventional suppository bases such as cocoa butter and other glycerides) or retention enemas for rectal delivery.

Therapeutic compounds that are or include nucleic acids can be administered by any method suitable for administration of nucleic acid agents, such as a DNA vaccine. These methods include gene guns, bio injectors, and skin patches as well as needle-free methods such as the micro-particle DNA vaccine technology disclosed in U.S. Pat. No. 6,194,389, and the mammalian transdermal needle-free vaccination with powder-form vaccine as disclosed in U.S. Pat. No. 6,168,587. Additionally, intranasal delivery is possible, as described in, inter alia, Hamajima et al., Clin. Immunol. Immunopathol., 88(2), 205-10 (1998). Liposomes (e.g., as described in U.S. Pat. No. 6,472,375), microencapsulation, micelles, e.g., polyelectrolyte complex (PEC) micelles (e.g., as described in Kim et al., PEG conjugated VEGF siRNA for anti-angiogenic gene therapy, J. Control. Release. 2006 Jun. 3; [Epub ahead of print]) or polyion complex (PIC) micelles (e.g., as described in Oishi et al., J. Am. Chem. Soc. 127(6):1624-1625 (2005)), or biodegradable targetable microparticle delivery systems (e.g., as described in U.S. Pat. No. 6,471,996) can also be used.

In one embodiment, the therapeutic compounds are prepared with carriers that will protect the therapeutic compounds against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Such formulations can be prepared using standard techniques. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

The pharmaceutical compositions can be included in a container, pack, or dispenser together with instructions for administration.

Methods of Treatment

The methods described herein include methods for the treatment of RA. Generally, the methods include administering a therapeutically effective amount of therapeutic compound as described herein, to a subject who is in need of, or who has been determined to be in need of, such treatment.

As used in this context, to “treat” means to ameliorate at least one symptom of RA. Often, RA is associated with inflammation and joint pain, e.g., joint pain felt on both sides of the body, affecting the wrist, knees, elbows, fingers, toes, ankle and/or neck, limiting mobility; thus, a treatment can result in a reduction in inflammation and joint pain, and improved mobility, e.g., a return or approach to normal mobility.

Dosage, toxicity and therapeutic efficacy of the compounds can be determined, e.g., by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compounds that exhibit high therapeutic indices are preferred. While compounds that exhibit toxic side effects may be used, care should be taken to design a delivery system that targets such compounds to the site of affected tissue in order to minimize potential damage to uninfected cells and, thereby, reduce side effects.

The data obtained from the cell culture assays and animal studies can be used in formulating a range of dosage for use in humans. The dosage of such compounds lies preferably within a range of circulating concentrations that include the ED50 with little or no toxicity. The dosage may vary within this range depending upon the dosage form employed and the route of administration utilized. For any compound used in the method of the invention, the therapeutically effective dose can be estimated initially from cell culture assays. A dose may be formulated in animal models to achieve a circulating plasma concentration range that includes the IC50 (i.e., the concentration of the test compound that achieves a half-maximal inhibition of symptoms) as determined in cell culture. Such information can be used to more accurately determine useful doses in humans. Levels in plasma may be measured, for example, by high performance liquid chromatography.

An “effective amount” is an amount sufficient to effect beneficial or desired results. For example, a therapeutic amount is one that achieves the desired therapeutic effect. This amount can be the same or different from a prophylactically effective amount, which is an amount necessary to prevent onset of disease or disease symptoms. An effective amount can be administered in one or more administrations, applications or dosages. A therapeutically effective amount of a composition depends on the composition selected. The compositions can be administered one from one or more times per day to one or more times per week; including once every other day. The skilled artisan will appreciate that certain factors may influence the dosage and timing required to effectively treat a subject, including but not limited to the severity of the disease or disorder, previous treatments, the general health and/or age of the subject, and other diseases present. Moreover, treatment of a subject with a therapeutically effective amount of the compositions described herein can include a single treatment or a series of treatments.

Methods of Screening

The invention includes methods for screening of test compounds, to identify compounds that inhibit NMU or an NMU receptor. A number of assays are known in the art for identifying inhibitors of NMU signalling, e.g., binding and biochemical assays. See, e.g., Johnson et al., J. Immunol. 173(12):7230-7238 (2004). For example, binding assays can be conducted using cells having an NMU-R, e.g., NMU-R1, in the membrane, or a membrane preparation from such cells including the NMU-R. Calcium signalling can be measured in cells responsive to NMU, e.g., cells that express the NMU-R and can signal in response to NMU.

Primary High-Through-Put Methods for Screening Libraries of Peptide Fragments or Homologs

Various techniques are known in the art for screening peptides, e.g., synthetic peptides, e.g., small molecular weight peptides (e.g., linear or cyclic peptides) or generated mutant gene products. Techniques for screening large gene libraries often include cloning the gene library into replicable expression vectors, transforming appropriate cells with the resulting library of vectors, and expressing the genes under conditions in which detection of a desired activity, assembly into trimeric molecules, binding to natural ligands, e.g., a receptor or substrates, facilitates relatively easy isolation of the vector encoding the gene whose product was detected. Each of the techniques described below is amenable to high through-put analysis for screening large numbers of sequences created, e.g., by random mutagenesis techniques.

Two Hybrid Systems

Two hybrid (interaction trap) assays can be used to identify a protein that interacts with ARNT2. These can include, e.g., agonists, superagonists, and antagonists of ARNT2 (the subject protein and a protein it interacts with are used as the bait protein and fish proteins). These assays rely on detecting the reconstitution of a functional transcriptional activator mediated by protein-protein interactions with a bait protein. In particular, these assays make use of chimeric genes that express hybrid proteins. The first hybrid comprises a DNA-binding domain fused to the bait protein, e.g., ARNT2 or active fragments thereof. The second hybrid protein contains a transcriptional activation domain fused to a fish protein, e.g., an expression library. If the fish and bait proteins are able to interact, they bring into close proximity the DNA-binding and transcriptional activator domains. This proximity is sufficient to cause transcription of a reporter gene, which is operably linked to a transcriptional regulatory site, which is recognized by the DNA binding domain; and expression of the marker gene can be detected and used to score for the interaction of the bait protein with another protein.

Display Libraries

In one approach to screening assays, the candidate peptides are displayed on the surface of a cell or viral particle, and the ability of particular cells or viral particles to bind an appropriate receptor protein via the displayed product is detected in a “panning assay.” For example, the gene library can be cloned into the gene for a surface membrane protein of a bacterial cell, and the resulting fusion protein detected by panning (Ladner et al., WO 88/06630; Fuchs et al., Bio/Technology, 9:1370-1371 (1991); and Goward et al., TIBS, 18:136-140 (1992)). This technique was used in Sahu et al., J. Immunology, 157:884-891 (1996), to isolate a complement inhibitor. In a similar fashion, a detectably labeled ligand can be used to score for potentially functional peptide homologs. Fluorescently labeled ligands, e.g., receptors, can be used to detect homologs that retain ligand-binding activity. The use of fluorescently labeled ligands allows cells to be visually inspected and separated under a fluorescence microscope, or, where the morphology of the cell permits, to be separated by a fluorescence-activated cell sorter.

A gene library can be expressed as a fusion protein on the surface of a viral particle. For instance, in the filamentous phage system, foreign peptide sequences can be expressed on the surface of infectious phage, thereby conferring two significant benefits. First, since these phage can be applied to affinity matrices at concentrations well over 10¹³ phage per milliliter, a large number of phage can be screened at one time. Second, since each infectious phage displays a gene product on its surface, if a particular phage is recovered from an affinity matrix in low yield, the phage can be amplified by another round of infection. The group of almost identical E. coli filamentous phage M13, fd., and fl are most often used in phage display libraries. Either of the phage gIII or gVIII coat proteins can be used to generate fusion proteins, without disrupting the ultimate packaging of the viral particle. Foreign epitopes can be expressed at the NH₂-terminal end of pIII and phage bearing such epitopes recovered from a large excess of phage lacking this epitope (Ladner et al., PCT publication WO 90/02909; Garrard et al., PCT publication WO 92/09690; Marks et al., J. Biol. Chem., 267:16007-16010 (1992); Griffiths et al., EMBO J., 12:725-734 (1993); Clackson et al., Nature, 352:624-628 (1991); and Barbas et al., Proc. Natl. Acad. Sci. USA, 89:4457-4461 (1992)).

A common approach uses the maltose receptor of E. coli (the outer membrane protein, LamB) as a peptide fusion partner (Charbit et al., EMBO, 5:3029-3037 (1986)). Oligonucleotides have been inserted into plasmids encoding the LamB gene to produce peptides fused into one of the extracellular loops of the protein. These peptides are available for binding to ligands, e.g., to antibodies, and can elicit an immune response when the cells are administered to animals. Other cell surface proteins, e.g., OmpA (Schorr et al., Vaccines, 91:387-392 (1991)), PhoE (Agterberg et al., Gene, 88:37-45 (1990)), and PAL (Fuchs et al., Bio/Tech., 9:1369-1372 (1991)), as well as large bacterial surface structures have served as vehicles for peptide display. Peptides can be fused to pilin, a protein which polymerizes to form the pilus-a conduit for interbacterial exchange of genetic information (Thiry et al., Appl. Environ. Microbiol., 55:984-993 (1989)). Because of its role in interacting with other cells, the pilus provides a useful support for the presentation of peptides to the extracellular environment. Another large surface structure used for peptide display is the bacterial motive organ, the flagellum. Fusion of peptides to the subunit protein flagellin offers a dense array of may peptides copies on the host cells (Kuwajima et al., Bio/Tech., 6:1080-1083 (1988)). Surface proteins of other bacterial species have also served as peptide fusion partners. Examples include the Staphylococcus protein A and the outer membrane protease IgA of Neisseria (Hansson et al., J. Bacteriol., 174:4239-4245 (1992) and Klauser et al., EMBO J., 9:1991-1999 (1990)).

In the filamentous phage systems and the LamB system described above, the physical link between the peptide and its encoding DNA occurs by the containment of the DNA within a particle (cell or phage) that carries the peptide on its surface. Capturing the peptide captures the particle and the DNA within. An alternative scheme uses the DNA-binding protein Lad to form a link between peptide and DNA (Cull et al., Proc. Natl. Acad. Sci. USA, 89:1865-1869 (1992)). This system uses a plasmid containing the Lad gene with an oligonucleotide cloning site at its 3′-end. Under the controlled induction by arabinose, a LacI-peptide fusion protein is produced. This fusion retains the natural ability of Lad to bind to a short DNA sequence known as LacO operator (LacO). By installing two copies of LacO on the expression plasmid, the LacI-peptide fusion binds tightly to the plasmid that encoded it. Because the plasmids in each cell contain only a single oligonucleotide sequence and each cell expresses only a single peptide sequence, the peptides become specifically and stably associated with the DNA sequence that directed its synthesis. The cells of the library are gently lysed and the peptide-DNA complexes are exposed to a matrix of immobilized receptor to recover the complexes containing active peptides. The associated plasmid DNA is then reintroduced into cells for amplification and DNA sequencing to determine the identity of the peptide ligands. As a demonstration of the practical utility of the method, a large random library of dodecapeptides was made and selected on a monoclonal antibody raised against the opioid peptide dynorphin B. A cohort of peptides was recovered, all related by a consensus sequence corresponding to a six-residue portion of dynorphin B. (Cull et al., Proc. Natl. Acad. Sci. USA, 89:1869 (1992))

This scheme, sometimes referred to as peptides-on-plasmids, differs in two important ways from the phage display methods. First, the peptides are attached to the C-terminus of the fusion protein, resulting in the display of the library members as peptides having free carboxy termini. Both of the filamentous phage coat proteins, pIII and pVIII, are anchored to the phage through their C-termini, and the guest peptides are placed into the outward-extending N-terminal domains. In some designs, the phage-displayed peptides are presented right at the amino terminus of the fusion protein. (Cwirla, et al., Proc. Natl. Acad. Sci. USA, 87:6378-6382 (1990)) A second difference is the set of biological biases affecting the population of peptides actually present in the libraries. The LacI fusion molecules are confined to the cytoplasm of the host cells. The phage coat fusions are exposed briefly to the cytoplasm during translation but are rapidly secreted through the inner membrane into the periplasmic compartment, remaining anchored in the membrane by their C-terminal hydrophobic domains, with the N-termini, containing the peptides, protruding into the periplasm while awaiting assembly into phage particles. The peptides in the Lad and phage libraries may differ significantly as a result of their exposure to different proteolytic activities. The phage coat proteins require transport across the inner membrane and signal peptidase processing as a prelude to incorporation into phage. Certain peptides exert a deleterious effect on these processes and are underrepresented in the libraries (Gallop et al., J. Med. Chem., 37(9):1233-1251 (1994)). These particular biases are not a factor in the Lad display system.

The number of small peptides available in recombinant random libraries is enormous. Libraries of 10⁷-10⁹ independent clones are routinely prepared. Libraries as large as 10¹¹ recombinants have been created, but this size approaches the practical limit for clone libraries. This limitation in library size occurs at the step of transforming the DNA containing randomized segments into the host bacterial cells. To circumvent this limitation, an in vitro system based on the display of nascent peptides in polysome complexes has recently been developed. This display library method has the potential of producing libraries 3-6 orders of magnitude larger than the currently available phage/phagemid or plasmid libraries. Furthermore, the construction of the libraries, expression of the peptides, and screening, is done in an entirely cell-free format.

In one application of this method (Gallop et al., J. Med. Chem., 37(9):1233-1251 (1994)), a molecular DNA library encoding 10¹² decapeptides was constructed and the library expressed in an E. coli S30 in vitro coupled transcription/translation system. Conditions were chosen to stall the ribosomes on the mRNA, causing the accumulation of a substantial proportion of the RNA in polysomes and yielding complexes containing nascent peptides still linked to their encoding RNA. The polysomes are sufficiently robust to be affinity purified on immobilized receptors in much the same way as the more conventional recombinant peptide display libraries are screened. RNA from the bound complexes is recovered, converted to cDNA, and amplified by PCR to produce a template for the next round of synthesis and screening. The polysome display method can be coupled to the phage display system. Following several rounds of screening, cDNA from the enriched pool of polysomes can be cloned into a phagemid vector. This vector serves as both a peptide expression vector, displaying peptides fused to the coat proteins, and as a DNA sequencing vector for peptide identification. By expressing the polysome-derived peptides on phage, one can either continue the affinity selection procedure in this format or assay the peptides on individual clones for binding activity in a phage ELISA, or for binding specificity in a completion phage ELISA (Barret et al., Anal. Biochem., 204:357-364 (1992)). To identify the sequences of the active peptides one sequences the DNA produced by the phagemid host.

Secondary Screens

The high through-put assays described above can be followed (or substituted) by secondary screens in order to identify biological activities which will, e.g., allow one skilled in the art to differentiate agonists from antagonists. The type of a secondary screen used will depend on the desired activity that needs to be tested. For example, glucose tolerance and insulin secretion assays described herein can be used, in which the ability to modulate, e.g., decrease or increase expression, level, or activity of ARNT2 in pancreatic islet beta cells can be used to identify ARNT2 agonists and antagonists from a group of peptide fragments isolated though one of the primary screens described above.

In some embodiments, the methods include administering a test compound identified as an inhibitor of NMU signalling, e.g., by a method described herein, to the K/BxN T cell receptor (TCR) transgenic model of arthritis, and evaluating clinical parameters associated with RA in the model to determine whether the test compound may be an effective therapeutic agent.

The K/BxN model of arthritis was discovered when a TCR transgenic mouse (KRN on the C57BL/6 background) was crossed with a non-obese diabetic (NOD) mouse (Kouskoff et al., Cell 87:811-822 (1996)). All of the offspring developed arthritis with many features of human RA. Since the initial description of the model, much has been learned about the mechanisms of autoimmune pathogenesis.

The development of arthritis in the K/BxN model occurs in two phases: the initiation phase and the effector phase. During both phases, the key autoantigen is glucose-6-phosphate isomerase (GPI), a ubiquitously expressed glycolytic enzyme (Matsumoto et al., Science 286:1732-35 (1999)). During the initiation phase of arthrits, CD4+ T cells expressing the KRN TCR recognize a peptide derived from GPI and presented by the NOD-derived class II major histocompatibility complex (MHC) allele I-A^{g7} (Matsumoto et al., Science 286:1732-35 (1999); Korganow et al., Immunity 10:451-61 (1999); Maccioni et al., J Exp Med 195:1071-77 (2002)). (In fact, I-A^{g7} is the only NOD-derived gene required for arthritis induction—all other NOD alleles are unnecessary.) These activated KRN T cells then provide help to GPI-specific B cells, resulting in the production of GPI-specific autoantibodies in high titer, mostly IgG1. Thus, the initiation phase depends on the breakdown of both T and B cell tolerance to GPI.

Passive transfer of GPI-specific autoantibodies contained in serum from arthritic K/BxN mice is sufficient to cause arthritis, even in recipient mice lacking T and B cells (Korganow et al., Immunity 10:451-61 (1999)). Thus, the effector phase of arthritogenesis in this model is driven the innate immune system. The critical players of the innate immune system have been identified by serum transfer experiments into recipient mice genetically lacking specific components of the innate immune system or depleted of specific cell populations. The key effectors of arthritis in the serum transfer model include neutrophils and mast cells, the cytokines TNF and IL-1, the alternative pathway of complement activation, C5a and its receptor, the low affinity IgG receptor FcγRIII, the neonatal Fc receptor (FcRn), and the adhesion molecule LFA-1 (Wipke and Allen, J Immunol 167:1601-1608 (2001); Corr and Crain, J Immunol 169:6604-9 (2002); Ji et al., Immunity 16:157-68 (2002); Ji et al., J Exp Med 196:77-85 (2002); Lee et al., Science 297:1689-92 (2002); Akilesh et al., J Clin Invest 113:1328-33 (2004)). Not required for arthritogenesis are the classical pathway of complement activation and the high-affinity receptors for IgG (FcγRI) (Ji et al., Immunity 16:157-68 (2002)) and IgE (FIER1).

Test Composunds

In some embodiments, the test compounds are initially members of a library, e.g., an inorganic or organic chemical library, peptide library, oligonucleotide library, or mixed-molecule library. In some embodiments, the methods include screening small molecules, e.g., natural products or members of a combinatorial chemistry library. As used herein, “small molecules” refers to small organic or inorganic molecules of molecular weight below about 3,000 Daltons.

A given library can comprise a set of structurally related or unrelated test compounds. Preferably, a set of diverse molecules should be used to cover a variety of functions such as charge, aromaticity, hydrogen bonding, flexibility, size, length of side chain, hydrophobicity, and rigidity. Combinatorial techniques suitable for creating libraries are known in the art, e.g., methods for synthesizing libraries of small molecules, e.g., as exemplified by Obrecht and Villalgordo, Solid-Supported Combinatorial and Parallel Synthesis of Small-Molecular-Weight Compound Libraries, Pergamon-Elsevier Science Limited (1998). Such methods include the “split and pool” or “parallel” synthesis techniques, solid-phase and solution-phase techniques, and encoding techniques (see, for example, Czarnik, Curr. Opin. Chem. Bio. 1:60-6 (1997)). In addition, a number of libraries, including small molecule libraries, are commercially available.

In some embodiments, the test compounds are peptide or peptidomimetic molecules, e.g., peptide analogs including peptides comprising non-naturally occurring amino acids or having non-peptide linkages; peptidomimetics (e.g., peptoid oligomers, e.g., peptoid amide or ester analogues, β-peptides, D-peptides, L-peptides, oligourea or oligocarbamate); small peptides (e.g., pentapeptides, hexapeptides, heptapeptides, octapeptides, nonapeptides, decapeptides, or larger, e.g., 20-mers or more); cyclic peptides; other non-natural or unnatural peptide-like structures; and inorganic molecules (e.g., heterocyclic ring molecules). In some embodiments, the test compounds are nucleic acids, e.g., DNA or RNA oligonucleotides.

The libraries useful in the methods of the invention can include the types of compounds that will potentially bind to NMU or an NMU-R, e.g., NMU-R1. For example, the test compounds can be structurally similar to NMU, but not activate signalling through the NMU-R.

In some embodiments, test compounds and libraries thereof can be obtained by systematically altering the structure of a first test compound. Taking a small molecule as an example, e.g., a first small molecule is selected that is, e.g., structurally similar to NMU, or has been identified as capable of binding NMU or an NMU-R, e.g., NMU-R1. For example, in one embodiment, a general library of small molecules is screened, e.g., using the methods described herein, to select a fist test small molecule. Using methods known in the art, the structure of that small molecule is identified if necessary and correlated to a resulting biological activity, e.g., by a structure-activity relationship study. As one of skill in the art will appreciate, there are a variety of standard methods for creating such a structure-activity relationship. Thus, in some instances, the work may be largely empirical, and in others, the three-dimensional structure of an endogenous polypeptide or portion thereof can be used as a starting point for the rational design of a small molecule compound or compounds.

In some embodiments, test compounds identified as “hits” (e.g., test compounds that demonstrate binding to NMU or an NMU-R, e.g., NMU-R1 and significantly decrease NMU signaling) in a first screen are selected and optimized by being systematically altered, e.g., using rational design, to optimize binding affinity, avidity, specificity, or other parameter. Such potentially optimized structures can also be screened using the methods described herein. Thus, in one embodiment, the invention includes screening a first library of test compounds using a method described herein, identifying one or more hits in that library, subjecting those hits to systematic structural alteration to create one or more second generation compounds structurally related to the hit, and screening the second generation compound. Additional rounds of optimization can be used to identify a test compound with a desirable therapeutic profile.

Test compounds identified as hits can be considered candidate therapeutic compounds, useful in treating disorders described herein. Thus, the invention also includes compounds identified as “hits” by a method described herein, and methods for their administration and use in the treatment, prevention, or delay of development or progression of a disease described herein, e.g., RA.

### EXAMPLES

The invention is further described in the following examples, which do not limit the scope of the invention described in the claims.

### Example 1

To determine whether NMU-deficient mice are resistant to serum-transfer arthritis in the K/BxN system, 200 mL of serum from K/BxN arthritic mice was injected intravenously into control B6 (NMU+/+) mice and NMU knockout mice (NMU−/−) on day 0. Arthritis scores (maximum=12) and ankle measurements were obtained for two weeks.

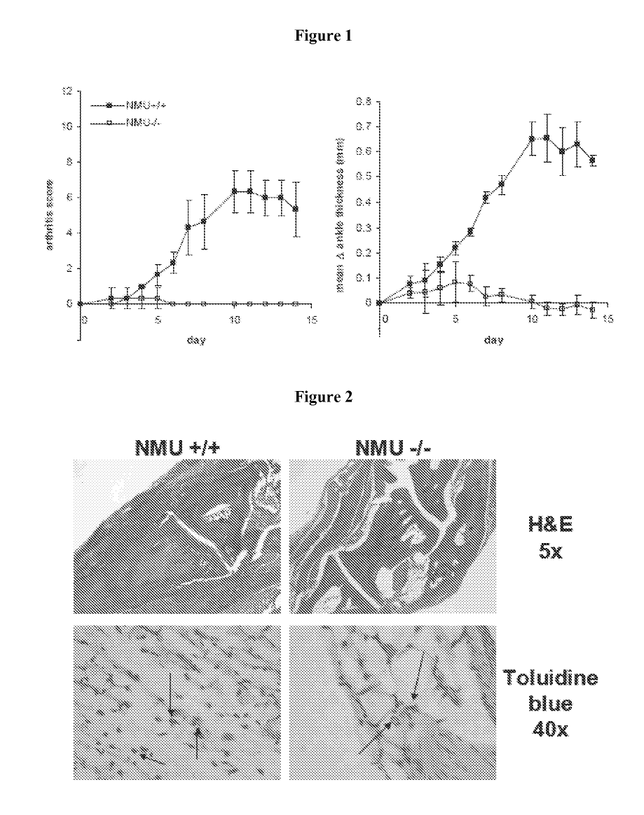

As shown in FIG. 1, NMU-deficient mice are resistant to serum-transfer arthritis in the K/BxN system. This finding suggests that NMU plays a role in the effector phase of arthritogenesis.

### Example 2

Histologic examination of the ankles from NMU knockout mice demonstrate essentially no signs of inflammation. Given the role of mast cells in the development of serum transfer arthritis, it was verified that NMU-deficient mice do express mast cells in and around the joints. Ankles were removed from control B6 (NMU+/+) mice and NMU −/− mice 14 days following injection of 200 mL of arthritogenic serum. The ankles were sectioned and stained with hematoxylin and eosin (H&E) (FIG. 2, top panels).

As shown in FIG. 2, Toluidine blue staining demonstrates the presence of mast cells in NMU −/− mice (arrows in lower panels).

### Example 3

To determine whether early activation of mast cells is intact in NMU-deficient mice, early vascular leak response following injection of arthritogenic serum was evaluated using intravital confocal microscopy as described in Binstadt et al., Nat. Immunol. 7(3):284-92 (2006), Epub 2006 Jan. 29.

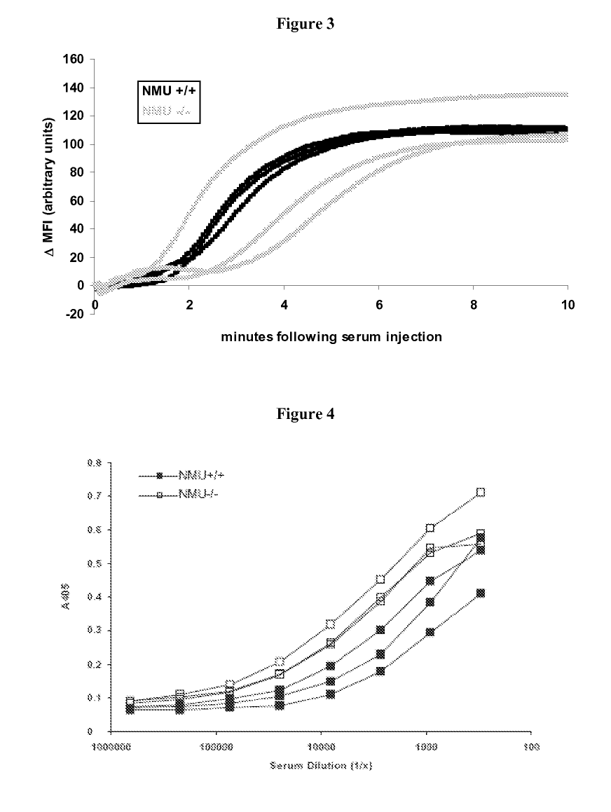

The results, shown in FIG. 3, indicated that the early vascular leak response was normal, suggesting that early activation of mast cells is intact.

### Example 4

One possible explanation for the resistance of NMU-deficient mice to the development of serum transfer arthritis could be that these mice metabolize the arthritogenic antibodies more quickly, similar to the mechanism by which mice lacking the neonatal Fc receptor, FcRn, are resistant to arthritis in this model.

To evaluate this hypothesis, serum was collected from control B6 (NMU+/+) mice and NMU −/− mice 14 days following injection of 200 mL of arthritogenic serum (n=3 mice/group). 3-fold dilutions of each serum sample were assayed for the presence of anti-glucose-6-phosphate isomerase (GPI) IgG with a standard ELISA utilizing plate-bound recombinant GPI and alkaline phosphatase-coupled secondary antibodies for detection.

The results, shown in FIG. 4, indicated that NMU-deficient mice maintain titers of the pathogenic anti-GPI IgG equivalent to control animals.

In sum, these findings demonstrate that NMU plays a critical role in the development of inflammatory arthritis. The experiments further suggest that the mechanism by which NMU deficiency impairs the development of arthritis in this model involves neither the impairment of the development or function of mast cells nor enhanced clearance of the arthritogenic antibodies.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A method of treating rheumatoid arthritis (RA) in an animal, the method comprising administering a therapeutically effective amount of a neuromedin U (NMU)-specific inhibitory nucleic acid.

2. The method of claim 1, wherein the inhibitory nucleic acid is selected from the group consisting of an NMU-specific siRNA, an NMU-specific antisense, an NMU-specific aptamer, and an NMU-specific ribozyme.

3. A method of treating rheumatoid arthritis (RA) in an animal, the method comprising administering a therapeutically effective amount of a neuromedin U (NMU) inhibitory peptide comprising the sequence Phe-Arg-Pro-Arg-Asn (SEQ ID NO:5).

4. A method of treating rheumatoid arthritis (RA) in an animal, the method comprising administering a therapeutically effective amount of an antibody or antigen binding fragment thereof that binds to a neuromedin U Receptor (NMU-R) and inhibits NMU signalling.

5. The method of claim 4, wherein the antibody or antigen-binding fragment thereof binds to NMU-R1.

6. The method of claim 5, wherein binding of the antibody or antigen-binding fragment thereof inhibits binding of NMU to the NMU-R1

7. A method of identifying a candidate compound for the treatment of rheumatoid arthritis (RA), the method comprising:
providing a sample comprising neuromedin U (NMU) and a NMU Receptor (NMU-R);
contacting the sample with a test compound; and
evaluating binding of the NMU to the NMU-R in the sample in the presence and absence of the test compound,
wherein a test compound that decreases binding of NMU to an NMU-R is a a candidate compound for the treatment of RA.

8. The method of claim 7, wherein the sample comprises a cell expressing the NMU-R.

9. The method of claim 7, wherein the sample comprises membranes isolated from cells expressing the NMU-R.

10. A method of identifying a candidate agent for the treatment of RA, the method comprising:
providing an experimental animal model of RA;
inducing RA in the model;
administering a candidate compound identified by the method of claim 7 to the animal before, during, and/or after inducing RA; and
evaluating one or more clinical parameters of RA in the animal model, wherein a candidate compound that is associated with an improvement in a clinical parameter of RA is a candidate agent for the treatment of RA.


SEQUENCE LISTING (5 sequences)

SEQ ID NO: 1 - protein, 174 aa, Homo sapiens
MLRTESCRPRSPAGQVAAASPLLLLLLLLAWCAGACRGAPILPQGLQPEQQLQLWNEIDDTCSSFLSIDSQPQASNALEELCFMIMGMLPKPQEQDEKDNTKRFLFHYSKTQKLGKSNVVSSVVHPLLQLVPHLHERRMKRFRVDEEFQSPFASQSRGYFLFRPRNGRRSAGFI

SEQ ID NO: 2 - protein, 25 aa, Homo sapiens
FRVDEEFQSPFASQSRGYFLFRPRN

SEQ ID NO: 3 - protein, 426 aa, Homo sapiens
MTPLCLNCSVLPGDLYPGGARNPMACNGSAARGHFDPEDLNLTDEALRLKYLGPQQTELFMPICATYLLIFVVGAVGNGLTCLVILRHKAMRTPTNYYLFSLAVSDLLVLLVGLPLELYEMWHNYPFLLGVGGCYFRTLLFEMVCLASVLNVTALSVERYVAVVHPLQARSMVTRAHVRRVLGAVWGLAMLCSLPNTSLHGIQQLHVPCRGPVPDSAVCMLVRPRALYNMVVQTTALLFFCLPMAIMSVLYLLIGLRLRRERLLLMQEAKGRGSAAARSRYTCRLQQHDRGRRQVTKMLFVLVVVFGICWAPFHADRVMWSVVSQWTDGLHLAFQHVHVISGIFFYLGSAANPVLYSLMSSRFRETFQEALCLGACCHRLRPRHSSHSLSRMTTGSTLCDVGSLGSWVHPLAGNDGPEAQQETDPS

SEQ ID NO: 4 - protein, 415 aa, Homo sapiens
MSGMEKLQNASWIYQQKLEDPFQKHLNSTEEYLAFLCGPRRSHFFLPVSVVYVPIFVVGVIGNVLVCLVILQHQAMKTPTNYYLFSLAVSDLLVLLLGMPLEVYEMWRNYPFLFGPVGCYFKTALFETVCFASILSITTVSVERYVAILHPFRAKLQSTRRRALRILGIVWGFSVLFSLPNTSIHGIKFHYFPNGSLVPGSATFTVIKPMWIYNFIIQVTSFLFYLLPMTVISVLYYLMALRLKKDKSLEADEGNANIQRPCRKSVNKMLFVLVLVFAICWAPFHIDRLFFSFVEEWSESLAAVFNLVHVVSGVFFYLSSAVNPIIYNLLSRRFQAAFQNVISSFHKQWHSQHDPQLPPAQRNIFLTECHFVELTEDIGPQFPCQSSMHNSHLPTALSSEQMSRTNYQSFHFNKT

SEQ ID NO: 5 - protein, 5 aa, Homo sapiens
FRPRN